(19) United States
(12) Patent Application Publication
(10) Pub. No.: US 2012/0208877 A1
(43) Pub. Date: Aug. 16, 2012

(54) Skin treatment in antiseptic thin film form with silver

(75) Inventor: Thomas M. Raffy, Riverside, CA (US)

(73) Assignee: James C. Caillouette

(21) Appl. No.: 13/373,875

(22) Filed: Dec. 5, 2011

Related U.S. Application Data
(60) Provisional application No. 61/463,168, filed on Feb. 14, 2011.

(51) Int. Cl. A61K 31/28; A61P 31/12; A61P 31/04

(52) U.S. Cl. 514/495

(57) ABSTRACT

A skin treating and antibacterial composition, consisting essentially water, emollient, emulsifier, humectant, thickener, silver or silver citrate, and additional ingredients, and providing a superior hand lotion.

This application claims priority from provisional application Ser. No. 61/463,168 filed Feb. 14, 2011.

### BACKGROUND OF THE INVENTION

This invention relates generally to improvements in skin or hand lotion compositions, and more particularly to a superior lotion that provides decontamination such as antibacterial and/or antiviral protection to the skin of the user, as well as skin, smoothing and anti-roughness properties.

There is need for effectively protecting the hands, antiseptionally, in view of constant and/or repetitive hand contact with surfaces carrying unknown distribution of bacteria—as for example door knobs, door opening devices, vehicle steering wheels, tools, and other equipment.

### SUMMARY OF THE INVENTION

The present invention relates to an improved lotion, such as an antiseptic general use lotion composition applicable to the skin, as for example a hand lotion. IT is particularly characterized as antibacterial such as anti E. coli cells, and antiviral. As will be seen, it incorporates small amounts (less than 1% by weight silver or silver compound, such as silver citrate, in combination with lotion ingredients including water, an emollient, an emulsifier, an humectant, a thickener, together with less than 1% by weight of additional substances, as will be seen. Silver ion is typically released in the combination, for anti-microbial action.

The invention provides lotion properties of advantage over alternative lotions lacking durable antibacterial cell and/or antiviral properties as well as the particular combination of functions as disclosed herein.

### DETAILED DESCRIPTION

The preferred skin treatment anti-bacterial composition of the invention employs the following ingredients, with percentages by weight, as indicated.

| | % by weight |
|---|---|
| Water (Aqua) | 30.00-100.00 |
| Isopropyl Palmitate (Palm Oil) | 3.00-10.00 |
| Caprylic/Capric Triglyceride (Plant Derived) | 3.00-10.00 |
| Glyceryl Stearate (Plant Derived) | 3.00-10.00 |
| Glycerin (Plant Derived) | 1.00-3.00 |
| Cetearyl Alcohol (Plant Derived) | 1.00-3.00 |
| Butyrospermum Parkii (Shea Butter) Fruit | 1.00-3.00 |
| Polysorbate-60 (Plant Derived) | 1.00-3.00 |
| Phenoxyethanol (Preservative) | 0.30-1.00 |
| Ethylhexylglycerin (Plant Derived Preservative) | 0.30-1.00 |
| Xanthan gum (Natural Thickener) | 0.10-0.30 |
| Citric Acid (Naturally Derived) | 0.01-0.10 |
| Silver Citrate | 0.10-0.30 |
| Tocopherol (Antioxidant) | 0.01-0.10 |
| Glycolic Acid | 0.01-0.10 |
| Aloe Barbadensis (Aloe Vera) Leaf Juice | 0.01-0.10 |
| Panthenol (Pro-Vitamin B5) | 0.01-0.10 |
| Lactis Proteinum (Milk Protein) | 0.01-0.10 |
| Melaleuca Alternifolia (Tea Tree) Leaf O | 0.01-0.10 |
| Leptospermum Scoparium (Manuka) Oil) | 0.01-0.10 |
| | 100.00 |

Functions of the ingredients are as follows:

| | Function | Cas # |
|---|---|---|
| Water (Aqua) | Solvent | 7732-18-5 |
| Isopropyl Palmitate (Palm Oil) | Emollient | 142-91-6 |
| Caprylic/Capric Triglyceride (Plant Derived) | Emollient | 65381-09-1 |
| Glyceryl Stearate (Plant (Derived) | Emulsifier | 123-94-4 |
| Glycerin (Plant Derived) | Humectant | 56-81-5 |
| Cetearyl Alcohol (Plant (Derived) | Thickener | 5-44-5 |
| Butyrospermum Parkii (Shea Butter) Fruit | Emollient | 194043-92-0 |
| Polysorbate-60 (Plant Derived) | Emulsifier | 9005-67-8 |
| Phenoxyethanol (Preservative) | Preservative | 122-99-6 |
| Ethylhexylglycerin (Plant Derived) | Preservative | 70445-33-9 |
| Xantham Gum (Natural Thickener) | Thickener | 11138-66-2 |
| Citric Acid (Naturally Derived) | Acidifier | 77-92-2 |
| Silver Citrate | Buffer | No CAS # |
| Tocopherol (Antioxidant) | Antioxidant | 59-02-9 |
| Glycolic Acid | Acidifier | 79-14-1 |
| Aloe Barbadensis (Aloe Vera) Leaf Juice | Botanical | 8001-97-6 |
| Panthenol (Pro-Vitamin B5) | Moisturizer | 81-13-0 |
| Lactis Proteinum (milk protein) | Conditioner | 84082-51-9 |
| Melaleuca Alternifolia (Tea Tree) Leaf O | Anti-Dandrf | 68647-73-4 |
| Leptospermum Scoparium (Manuka) Oil | Anti-Dandrf | 219828-87-2 |

The indicated antiseptic property of silver citrate may be further characterized as antibacterial and/or antiviral, and adds significantly to the unusually effective skin treatment properties of the remaining ingredients, as combined.

The method of use of the above defined composition includes topically applying a sufficient amount of the composition to substantially cover the hand or hands of the user, as a thin film.

Such application is preferably at least once, on a daily basis, typically to remain on the hands as a protective film during the day.

The ingredients other than the dispersed silver ingredient assure thorough spreading of the silver ingredient onto and over the skin of the user, such as the hands, as a thin film, for effective treatment of the skin, including durable smoothing, as well as antibacterial and/or antiviral treatment or protection. The invention as disclosed is preferred, or in its preferred composition form.

I claim:

1. A skin treatment composition, consisting essentially of:
a) between 30% and 100% by weight water,
b) between 7% and 23% by weight emollient,
c) between 4% and 13% by weight emulsifier,
d) between 1.0% and 3.30% by weight thickener,
e) between 1.00% and 3.00% humectant,
f) between 0.01% and 2.30% by weight of ingredients selected from the group:
i) preservative
ii) acidifier
iii) antioxidant
iv) moisturizer,
v) botanical
vi) conditioner
vii) anti-dandrf
and g) between 0.10% and 0.30% by weight silver or silver compound.

2. The composition of claim 1 wherein said silver compound is silver citrate.

3. The composition of claim 1 wherein said emollient is selected from the group:
capryic/capric triglyceride
isopropyl palmitate
butyrospermum parkii.

4. The composition of claim 1 wherein said emulsifier is selected from the group:
glyceryl stearate (plant derived)
polysorbate-60 (plant derived).

5. The composite of claim 1 wherein said thickener is selected from the group:
xanthum gum
cetearyl alcohol.

6. The composition of claim 1 wherein said humectant is glyceric.

7. The composition of claim 1 including, or wherein:
said Acidifier is citric acid (naturally derived)
said silver compound is Silver Citrate
said Antioxidant is Tocopherol (Antioxidant)
including Acidifier which is Glycolic Acid including Botanical i.e. Aloe Barbadensis (Aloe Vera) Leaf Juice
including Moisturizer i.e. Panthenol (Pro-Vitamin B5)
said Conditioner is Lactis Proteinum (Milk Protein)
and including Anti-Dandrf which is Melaleuca Alternifolia (Tea Tree) Leaf O
including Anti-Dandrf which is Leptospermum Scoparium (Manuka) Oil

8. The composition of claim 3 wherein said silver compound is silver citrate.

9. The composition of claim 4 wherein said silver compound is silver citrate.

10. The composition of claim 7 wherein said silver compound is silver citrate.

11. The composition of claim 1 wherein said silver compound is silver citrate.

12. A skin treatment composition consisting essentially of the following in combination:

| % by wt. | |
|---|---|
| 30.00-100.00 | Water (Aqua) |
| 3.00-10.00 | Isopropyl Palmitate (Palm Oil0 |
| 3.00-10.00 | Caprylic/Capric Triglyceride (Plant Derived) |
| 3.00-10.00 | Glyceryl Stearate (Plant Derived) |
| 1.00-3.00 | Glycerin (Plant Derived) |
| 1.00-3.00 | Cetearyl Alcohol (Plant Derived) |
| 1.00-3.00 | Butyrospermum Parkii (Shea Butter) Fruit |
| 1.00-3.00 | Polysorbate-60 (Plant Derived) |
| 0.30-1.00 | Phenoxyethanol (Preservative) |
| 0.30-1.00 | Ethylhexylglycerin (Plant Derived Preservative) |
| 0.10-0.30 | Xantham Gum (Natural Thickener) |
| 0.10-0.30 | Citric Acid (Naturally Derived) |
| 0.10-0.30 | Silver Citrate |
| 0.01-0.10 | Tocopherol (Antioxidant) |
| 0.01-0.10 | Glycolic Acid |
| 0.01-0.10 | Aloe Barbadensis (Aloe Vera) Leaf Juice |
| 0.01-0.10 | Panthenol (Pro-Vitamin B5) |
| 0.01-0.10 | Lactis Proteinum (Milk Protein) |
| 0.01-0.10 | Melaleuca Alternifolia (Tea Tree) Leaf O |
| 0.01-0.10 | Leptospermum Scoparium (Manuka) Oil |
| 100.00 | |

13. A skin treatment composition consisting of:

| | Function | CAS# |
|---|---|---|
| Water (Aqua) | Solvent | 7732-18-5 |
| Isopropyl Palmitate (Palm Oil) | Emollient | 142-91-6 |
| Caprylic/Capric Triglyceride (Plant Derived) | Emollient | 65381-09-1 |
| Glyceryl Stearate (Plant (Derived) | Emulsifier | 123-94-4 |
| Glycerin (Plant Derived) | Humectant | 56-81-5 |
| Cetearyl Alcohol (Plant (Derived) | Thickener | 5-44-5 |
| Butyrospermum Parkii (Shea Butter) Fruit | Emollient | 194043-92-0 |
| Polysorbate-60 (Plant Derived) | Emulsifier | 9005-67-8 |
| Phenoxyethanol (Preservative) | Preservative | 122-99-6 |
| Ethylhexylglycerin (Plant Derived) | Preservative | 70445-33-9 |
| Xantham Gum (Natural Thickener) | Thickener | 11138-66-2 |
| Citric Acid (Naturally Derived) | Acidifier | 77-92-2 |
| Silver Citrate | Buffer | No CAS # |
| Tocopherol (Antioxidant) | Antioxidant | 59-02-9 |
| Glycolic Acid | Acidifier | 79-14-1 |
| Aloe Barbadensis (Aloe Vera) Leaf Juice | Botanical | 8001-97-6 |
| Panthenol (Pro-Vitamin B5) | Moisturizer | 81-13-0 |
| Lactis Proteinum (milk protein) | Conditioner | 84082-51-9 |
| Melaleuca Alternifolia (Tea Tree) Leaf O | Anti-Dandrf | 68647-73-4 |
| Leptospermum Scoparium (Manuka) Oil | Anti-Dandrf | 219828-87-2 |

14. The method of use of the spreadable composition of claim 1 that includes topically applying sufficient amount of the composition to substantially cover a major extent of the hand or hands of the user, as a thin film.

15. The method of use of the composition as defined in claim 14 wherein topical application thereof is at least once per day.

16. The composition that consists essentially of
a) a hand lotion,
b) silver citrate dispersed substantially uniformly in the lotion, to constitute between about 0.10 and 0.30 percent by weight of the lotion.

17. The method of use of the composition of claim 16 that includes topically applying sufficient amount of the composition to substantially cover a major extent of the hand or hands of the user, as a thin film.

18. The method of claim 17 wherein said application of the composition is at least once, on a daily basis.

19. The method of claim 17 wherein said composition includes:
said Acidifier is citric acid (naturally derived)
said silver compound is Silver Citrate
said Antioxidant is Tocopherol (Antioxidant)
including Acidifier which is Glycolic Acid including Botanical i.e. Aloe Barbadensis (Aloe Vera) Leaf Juice
including Moisturizer i.e. Panthenol (Pro-Vitamin B5)
said Conditioner is Lactis Proteinum (Milk Protein)
and including Anti-Dandrf which is Melaleuca Alternifolia (Tea Tree) Leaf O
including Anti-Dandrf which is Leptospermum Scoparium (Manuka) Oil

20. The method of use of the composition of claim 12 that includes topically applying sufficient amount of the composition to substantially cover a major extent of the hand or hands of the user, as a thin film.

21. The method of use of the composition of claim 13 that includes topically applying sufficient amount of the composition to substantially cover a major extent of the hand or hands of the user, as a thin film.
